(19) United States
(12) Patent Application Publication
(10) Pub. No.: US 2012/0128531 A1
(43) Pub. Date: May 24, 2012

(54) METHOD FOR DECONTAMINATION OF OBJECTS

(75) Inventors: Hermann Wolf Ibach, Schlesslitz (DE); Martin Pracher, Wuerzburg (DE)

(73) Assignee: HERMES HOLDING GMBH, ERKRATH (DE)

(21) Appl. No.: 13/387,745

(22) PCT Filed: Jul. 21, 2010
(86) PCT No.: PCT/EP2010/004449

§ 371 Date: Jan. 30, 2012

(30) Foreign Application Priority Data
Jul. 30, 2009 (DE) 10 2009 035 379.8

(51) Int. Cl. A61L 2/04

(52) U.S. Cl. 422/38; 422/1

(57) ABSTRACT

The invention relates to a method for decontamination of objects such as art and cultural objects and work materials, in order to remove undesired, particularly to remove toxic, substances contained in the object, wherein a temperature above ambient temperature is created and/or a lower or higher pressure than ambient pressure is created, in a treatment chamber surrounding the objects.

### FIELD OF THE INVENTION

The invention relates to a method for decontamination of objects such as art- and cultural-objects and work materials, in order to remove undesired, particularly toxic, substances contained in the object.

### BACKGROUND

For protection of art- and cultural-goods, materials were used, until recently, which turned out later to be substances dangerous to health. The infused substances degrade in the course of time and burden the ambient air and neighboring objects. Depending upon the protective material employed—DDT, PCP, LINDANE or combinations thereof (e.g. HYLOTOX) and also mercury compound—the outgassing rate varies a lot. These materials are, to some extent, widely employed. The average absorption and penetration depth were, depending upon material and retention state, different from object to object.

In recent years, possibilities for removal of these damaging substances have been sought. The solutions found to the problem have, until now, not been used because they require, on the one hand, very expensive technology (e.g. application of supercritical CO₂) and, on the other hand, are seen as dangerous.

### SUMMARY OF THE INVENTION

It is an object of the invention to provide a method for decontamination of objects such as art- and cultural-goods which involves simple and economical treatment, only slightly impacts the objects, and does not endanger the user and the environment, especially when toxic substances are present.

This object is achieved, in accordance with the invention, by creating, in a treatment chamber surrounding the object(s), a temperature elevated with respect to ambient temperature and/or a lower or higher pressure, relative to the ambient pressure.

Such a method makes it possible to substantially, and often completely, free objects such as art- and cultural-articles, particularly made of wood, textiles, paper, leather, plastics and stone, and all other natural and synthetic organic and inorganic art- and work-materials of burdening, especially toxic, substances, without the material of the objects being attacked, and chemically or physically changed. Furthermore, neither the users of the method, nor the environment, come into contact with the substances being removed.

### DETAILED DESCRIPTION

Advantageously, it is proposed that the elevated temperature be 25 to 90 degrees, preferably 30 to 60 degrees Celsius. Further, the lowered pressure should be 900 to 100 mbar, preferably 500 to 200 mbar. By application of underpressure, there occurs an accelerated outgassing at object-preserving low temperatures, which otherwise would only occur at material-degrading high temperatures.

An acceleration and substantially improvement of the method is also achieved if, alternatively, an overpressure is created in the treatment chamber. Here, the reduced pressure should be carried out fluctuatingly, particularly interrupted by normal- and/or high-pressure phases.

Release of the undesired substances in the object is made easier by creating, in the treatment chamber, a relative humidity, applied on or in the object, of 30to 80%, preferably 35 to 75%. It is particularly advantageous if the relative humidity is kept constant during the under-pressure, normal-pressure and over-pressure phases by direct humidification by spraying and/or by indirect humidification and dehumidification using moisture-absorbing salts and gels. Also, the absolute humidity level can fluctuate during the under-pressure, normal-pressure and over-pressure phases, both in the treatment chamber and in the object, and this can lead to material agitation, going into solution, and mobilization of undesired, particularly of toxic, substances. Further, it is advantageous if an underpressure is applied to, or in, the object, while the object is in an autoclave with overpressure, and a continuous air flow through the substrate in one direction is thereby created. Advantageously, it is proposed that an overpressure is applied on, or in, the object, while the object is in an autoclave with underpressure, and a continuous air flow through the substrate in one direction is thereby created.

A burdening of the environment and of the user is avoided, if an absorber and/or an adsorber material is arranged in the treatment chamber, with which the undesired, especially the toxic, substances leaving the object are captured. Here, the absorber and/or adsorber material can comprises activated charcoal.

Advantageously, it is proposed that the treatment chamber be hermetically sealed with respect to ambient atmosphere. Also, the method can be particularly easily and technically reliably performed if the treatment chamber comprises an autoclave.

It is particularly advantageous, if the used air of the treatment chamber is regularly, especially continuously or discontinuously, sucked out by a circulation or ventilation system and cleaned through filters. It is further proposed that the air within the treatment chamber is circulated during the treatment process.

Advantageously, prior to, during and/or subsequent to treatment, one measures values of the air within the treatment chamber, and/or of the exhausted air, and/or of the object, especially the decontamination degree of the object and/or of the absorber- and/or adsorber material. Here, according to the values measured, the process can be controlled, and especially can be automatically controlled, so that the treatment duration is determined according to the measured values.

Advantageous exemplary examples of the method of the invention are described in more detail in the following.

The method for decontamination relates to a combination of method steps which, together with a suitable measurement process, lead to a substantial reduction—up to complete purification—of the contaminated art- and cultural-goods, without burdening the objects by the cleansing process more than absolutely necessary.

The, in part, very low vapor pressure of the toxic substances which have become embedded in the objects, such as, e.g. BIOZIDE, is taken into account by the fact that the objects, in a suitably dimensioned autoclave, are subjected to an underpressure of 900 mbar to 100 mbar, preferably of 500 to 200 mbar and simultaneously a temperature course happens in the autoclave which can be tolerated by the object(s) in the autoclave. The temperature range for wood is from 30 to 70 degrees, fabric 20 to 70 degrees, leather 20 to 60 degrees, taxidermy preparations 30 to 70 degrees Celsius. Material-preserving treatment includes allocating sufficiently long time spans for the application of underpressure, for the temperature elevation and for the “dwell time.” Often, it is not the goal of the method to achieve the removal of the toxic substances within a few minutes or hours, but rather in days and weeks, even multiple months as duration, according to the values measured in the treatment chamber air, in the circulated air and/or in the object.

Instead of, or supplemental to, an underpressure, one can also work in the treatment chamber/autoclaves with overpressure. Here, the overpressure is in the range 1 to 20 bar, advantageously 2 to 9 bar. Further, advantageously during the under-pressure, normal-pressure and high-pressure phases, the relative humidity is kept constant by direct humidification by spraying and/or indirect humidification and dehumidification using moisture-absorbing salts and gels. The absolute humidity fluctuates during the under-pressure, normal-pressure and high-pressure phases, both in the treatment chamber and in the object, and this leads to material agitation, going into solution, and mobilization of undesired, especially of toxic, substances. While the object is located in the autoclave with overpressure and thereby a continuous air flow through the substrate occurs in one direction, an underpressure is applied at, or in, the object. Alternatively, an overpressure is applied at, or in, the object while the object is located in the autoclave with underpressure and thereby a continuous air flow through the substrate occurs in one direction.

The climatic relationships in the autoclave are matched to the object to be treated. Thus, for example, during treatment of wood, a sufficiently moist climate of 30 to 65% relative humidity is created and continuously controlled.

The aforementioned relative humidities are also maintained during the pressure- or vacuum-phases.

In the under-pressure phase, the relative humidity is maintained by direct (spraying) or indirect (conditioning salts and silica gels). In the pressure phase, the relative humidity is indirectly maintained by moisture-absorbing conditioning salts.

By alternating pressure- and vacuum-phases, a continuous variation of the absolute humidity in the art- and work-material occurs. Further, there occurs a continuous air movement in the object, which leads to supplemental mobilization and out-gassing of the undesired, especially of the toxic, substances.

In an extension of the method for special cases, one applies vacuum against the object using a bell, or a vacuum inside the object using a bore, while the art- or work-material is placed in an autoclave under pressure. The thus-resulting continuous air draw or air washing leads to a strong elevation of the outgassing rate. Conversely, one could apply overpressure against the object using a bell or overpressure inside the object using a bore, while the object is in an autoclave with underpressure.

The air flow is, in each case, fed via suitable filter systems.

For specific objects, a temporary pressure increase can be carried out, to increase the take-up capacity of the surrounding air in the autoclave.

The inner sheathing of the autoclave can, in order to increase the separation rate of the toxic substances, include adsorbers and/or absorbers, whose take-up readiness and take-up capacity are matched to the stuff to be dissolved out.

The autoclaves installed for this purpose are equipped with an air circulation system, which increases the air quantity fed past the objects to be decontaminated and builds up a continuous air circulation, which further increases the outgassing rate at the object surface. Additionally, or alternatively, the air within the autoclave is circulated by ventilator(s).

Using tubular conduits which lead out of and back into the autoclave, in which a ventilator and a filter system are installed, the air within the autoclave is continuously freed of the toxic substances which have come out of the objects.

By means of the aforementioned adsorber and/or absorber systems and by means of the aforementioned filter, an environment-friendly disposal of the dissolved-out substances is possible.

1. A method for decontamination of objects such as art and cultural objects and work materials, in order to remove undesired contaminants contained therein,
characterized in that
a treatment chamber surrounds the object or objects,
a higher-than-ambient temperature is applied and/or
relative to ambient pressure, a lower or higher pressure is applied in the chamber.

2. The method of claim 1, characterized in that
the higher temperature is elevated about 25 to 90 degrees.

3. The method of claim 1, characterized in that
the lower pressure is 100 to 900 mbar lower than ambient.

4. The method of claim 1, characterized in that the lower pressure is fluctuatingly applied, and is interrupted by normal-pressure or high-pressure phases.

5. The method according to claim 1, characterized in that the elevation in pressure is
in the range of 1 to 20 bar, preferably in the range of 2-9 bar.

6. The method according to claim 1, characterized in that
within the treatment chamber, a predetermined relative humidity in the range 30 to 80% is applied to the object or objects.

7. The method according to claim 1,
characterized in that
during the underpressure, normal pressure and overpressure phases, the relative air humidity is kept constant by direct humidification by spraying and/or by indirect humidification and dehumidification using moisture-absorbing salts and gels.

8. The method according to claim 1,
characterized in that
during the underpressure, normal-pressure and overpressure phases, the absolute humidity, both in the treatment chamber and in the object(s), fluctuates and results in material agitation, going into solution and mobilization of the undesired contaminants.

9. The method according to claim 1,
characterized in that
an underpressure is applied on or within the object, while the object is located in an autoclave with elevated pressure, so that a continuous airflow occurs through the substrate in one direction.

10. The method according to claim 1,
characterized in that
an overpressure is applied on or within the object, while the object is located in an autoclave with diminished pressure, so that a continuous airflow occurs through the substrate in one direction.

11. The method according to claim 1,
characterized in that
an absorber and/or an adsorber is provided in the treatment chamber, enabling capture of the undesired contaminants emitted from the object(s).

12. The method according to claim 11,
characterized in that
the absorber and/or adsorber material comprises activated charcoal.

13. The method according to claim 1, characterized in that
the treatment chamber is hermetically sealed with respect to ambient atmosphere.

14. The method according to claim 1,
characterized in that
the treatment chamber is an autoclave.

15. The method according to claim 1,
characterized in that
the applied air of the treatment chamber is regularly, particularly continuously or discontinuously, withdrawn via a circulation system and purified through filters.

16. The method according to claim 1,
characterized in that
the air within the treatment chamber is circulated during the treatment.

17. The method according to claim 1,
further comprising
prior to, during, or after treatment,
the step of measuring at least one of
values of the air within the treatment chamber
values of exhaust air and
values of the object(s),
particularly a decontamination value of the object(s) and/or of the absorber- and/or adsorber material.

18. The method according to claim 17,
characterized in that
the method is automatically regulated, in accordance with the values measured.

19. The method according to claim 18,
characterized in that
duration of treatment is adjusted according to the values measured.

20. The method according to claim 6,
wherein said relative humidity is in the range 35% to 75%.
